(19) United States
(12) Patent Application Publication
(10) Pub. No.: US 2020/0268345 A1
(43) Pub. Date: Aug. 27, 2020

(54) ULTRASOUND PULSE SEQUENCES FOR CONTROLLABLE FRAME RATES AND ASSOCIATED DEVICES, SYSTEMS, AND METHODS

(71) Applicant: KONINKLIJKE PHILIPS N.V., EINDHOVEN (NL)

(72) Inventors: Francois Guy Gerard Marie VIGNON, ANDOVER, MA (US); Seungsoo KIM, ANDOVER, MA (US); Jun Seob SHIN, MEDFORD, MA (US); Jean-luc Francois-Marie ROBERT, CAMBRIDGE, MA (US); Andrew HANCOCK, SACRAMENTO, CA (US); David HOPE SIMPSON, BOTHELL, WA (US)

(21) Appl. No.: 16/798,502

(22) Filed: Feb. 24, 2020

Related U.S. Application Data
(60) Provisional application No. 62/808,889, filed on Feb. 22, 2019.

(51) Int. Cl. A61B 8/00; A61B 8/14; A61B 8/12

(52) U.S. Cl. CPC A61B 8/4488; A61B 8/4494; A61B 8/463; A61B 8/12; A61B 8/145

(57) ABSTRACT

Ultrasound systems and devices that perform scan sequences providing controllable and/or increased frame rates are provided. In one embodiment, an ultrasound system comprises an array of acoustic elements and a processor configured to perform a scan sequence comprising a plurality of transmit-receive pairs. The processor is configured to arrange the plurality of transmit-receive pairs into an aperture. The transmit-receive pairs corresponding to each transmit element correspond to only a portion of the plurality of receive elements and/or the transmit-receive pairs corresponding to each receive element correspond to only a portion of the plurality of transmit elements. Accordingly, fewer transmit-receive pairs are activated leading to faster aperture acquisitions, and therefore faster frame rates. Furthermore, the faster frame rates can be achieved without significant degradations in image quality.

CROSS-REFERENCE TO PRIOR APPLICATION

This application claims the benefit of U.S. Provisional Patent Application Ser. No. 62/808,889, filed Feb. 22, 2019, which is hereby incorporated by reference herein.

### TECHNICAL FIELD

The present disclosure relates generally to ultrasound imaging and, in particular, to Synthetic aperture ultrasound devices configured to perform pulse sequences images. For example, an ultrasonic medical imaging device can include an array of acoustic elements configured to perform a pulse sequence using accelerated aperture acquisition to generate images at increased frame rates.

### BACKGROUND

Intravascular ultrasound (IVUS) imaging is widely used in interventional cardiology as a diagnostic tool for assessing a diseased vessel, such as an artery, within the human body to determine the need for treatment, to guide the intervention, and/or to assess its effectiveness. An IVUS device including one or more ultrasound transducers is passed into the vessel and guided to the area to be imaged. The transducers emit ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a cross-sectional image of the vessel where the device is placed.

Solid-state (also known as synthetic-aperture) IVUS catheters are one of the two types of IVUS devices commonly used today, the other type being the rotational IVUS catheter. Solid-state IVUS catheters carry a scanner assembly that includes an array of ultrasound transducers positioned and distributed around its perimeter or circumference along with one or more integrated circuit controller chips mounted adjacent to the transducer array. The controllers select individual acoustic elements (or groups of elements) for transmitting a pulse of acoustic energy and for receiving the ultrasound echo signal corresponding to the transmitted ultrasound energy. By stepping through a sequence of transmit-receive pairs, the solid-state IVUS system can synthesize the effect of a mechanically scanned ultrasound transducer but without moving parts (hence the solid-state designation). Since there is no rotating mechanical element, the transducer array can be placed in direct contact with the blood and vessel tissue with minimal risk of vessel trauma. Furthermore, because there is no rotating element, the electrical interface is simplified. The solid-state scanner can be wired directly to the imaging system with a simple electrical cable and a standard detachable electrical connector, rather than the complex rotating electrical interface required for a rotational IVUS device.

IVUS devices can be used to generate a stream of images, providing a live view of the inside of a blood vessel. The stream of images is acquired based on a frame rate, measured in frames/second. The frame rates achievable by a solid state IVUS device are restricted by the scan sequence used to obtain the image stream. In some instances, a physician may desire to achieve higher frame rates than what are permitted by conventional scan sequences.

### SUMMARY

Aspects of the present disclosure provide ultrasound systems and devices that perform scan sequences providing controllable and/or increased frame rates. For example, in one embodiment, an ultrasound system comprises an array of acoustic elements and a processor configured to perform a scan sequence comprising a plurality of transmit-receive pairs arranged into apertures. For each aperture, transmit-receive pairs associated with any given transmit element of the aperture are associated with only a portion the receive elements of the aperture. Accordingly, fewer transmit-receive pairs are activated leading to faster aperture acquisitions, and therefore faster frame rates. Furthermore, in some aspects, the faster frame rates can be achieved without significant degradations in image quality.

According to one embodiment of the present disclosure, a system for ultrasound imaging includes an array of acoustic elements configured to transmit ultrasound energy into an anatomy and receive echoes associated with the transmitted ultrasound energy, and a processor circuit in communication with the array. The processor is configured to activate a pulse sequence comprising a plurality of transmit-receive pairs associated with a plurality of transmit elements and a plurality of receive elements of the array, and arrange the plurality of transmit-receive pairs into an aperture, the aperture spanning the plurality of transmit elements and the plurality of receive elements, wherein at least one of transmit-receive pairs corresponding to each transmit element correspond to only a portion of the plurality of receive elements; or transmit-receive pairs corresponding to each receive element correspond to only a portion of the plurality of transmit elements. The processor is further configured to generate an image using signals corresponding to the plurality of transmit-receive pairs arranged into the aperture, and output the image to a display in communication with the processor.

In some embodiments, the processor is configured to arrange the plurality of transmit-receive pairs into the aperture by beamforming signals associated with the plurality of transmit-receive pairs. In some embodiments, the processor is configured to arrange the plurality of transmit-receive pairs into the aperture by applying a weight to signals associated with each transmit-receive pair, wherein the applied weight is based on a distance between a transmit element and a receive element associated with the transmit-receive pair. In some embodiments, the processor is configured to activate the pulse sequence by activating the plurality of transmit-receive pairs and skipping one or more nonactivated transmit-receive pairs, wherein the one or more nonactivated transmit-receive pairs correspond to a lower signal-to-noise ratio than the plurality of transmit-receive pairs activated by the processor. In some aspects, the one or more nonactivated transmit-receive pairs span a range of spatial frequencies, and the plurality of transmit-receive pairs activated by the processor span at least the range of spatial frequencies. In another aspect, the processor is configured to activate the pulse sequence by activating each of the plurality of transmit-receive pairs more than once. In another aspect, arranging the plurality of transmit-receive pairs into an aperture comprises averaging duplicate transmit-receive pairs.

In some embodiments, the processor is configured to activate the pulse sequence by skipping one or more transmit-receive pairs and their spatial complements. In some embodiments, the system includes an intravascular ultrasound (IVUS) imaging catheter, and wherein the array of acoustic elements is positioned around a perimeter of the IVUS imaging catheter. In some aspects, the aperture comprises a contiguous group of transmit-receive pairs. In other aspects, the aperture comprises a non-contiguous group of transmit-receive pairs.

According to another embodiment of the present disclosure, a method includes transmitting, by an array of acoustic elements, ultrasound energy into an anatomy, receiving, by the array, echoes associated with the transmitted ultrasound energy, activating, by a processor circuit in communication with the array, a pulse sequence comprising a plurality of transmit-receive pairs associated with a plurality of transmit elements and a plurality of receive elements of the array, and arranging, by the processor, the plurality of transmit-receive pairs into an aperture, the aperture spanning the plurality of transmit elements and the plurality of receive elements, wherein at least one of: transmit-receive pairs corresponding to each transmit element correspond to only a portion of the plurality of receive elements; or transmit-receive pairs corresponding to each receive element correspond to only a portion of the plurality of transmit elements. The method further includes generating, by the processor, an image using signals corresponding to the plurality of transmit-receive pairs arranged into the aperture, and outputting the image to a display in communication with the processor.

In some embodiments, arranging the plurality of transmit-receive pairs into the aperture comprises beamforming signals associated with the plurality of transmit-receive pairs. In some embodiments, arranging the plurality of transmit-receive pairs into the aperture comprises applying a weight to signals associated with each transmit-receive pair, wherein the applied weight is based on a distance between a transmit element and a receive element associated with the transmit-receive pair. In some embodiments, activating the pulse sequence comprises activating the plurality of transmit-receive pairs, and skipping one or more nonactivated transmit-receive pairs, wherein the one or more nonactivated transmit-receive pairs correspond to a lower signal-to-noise ratio than the plurality of transmit-receive pairs activated by the processor.

In some aspects, the one or more nonactivated transmit-receive pairs span a range of spatial frequencies, wherein the plurality of transmit-receive pairs activated by the processor span at least the range of spatial frequencies. In another aspect, activating the pulse sequence comprises activating each of the plurality of transmit-receive pairs more than once, and arranging the plurality of transmit-receive pairs into the aperture comprises averaging duplicate transmit-receive pairs. In some embodiments, activating the pulse sequence comprises skipping one or more transmit-receive pairs and their spatial complements. In some embodiments, arranging the plurality of transmit-receive pairs into the aperture comprises arranging a contiguous group of transmit-receive pairs into the aperture. In some embodiments, arranging the plurality of transmit-receive pairs into the aperture comprises arranging a non-contiguous group of transmit-receive pairs into the aperture.

According to another embodiment of the present disclosure, a system comprises an array of acoustic elements configured to transmit ultrasound energy into an anatomy and receive echoes associated with the transmitted ultrasound energy, and a processor circuit in communication with the array. The processor is configured to activate a pulse sequence comprising a plurality of transmit-receive pairs associated with a plurality of transmit elements and a plurality of receive elements of the array, arrange the plurality of transmit-receive pairs into an aperture, wherein the aperture spans N transmit elements and N receive elements, and wherein the aperture comprises fewer than half of N(N+1) transmit-receive pairs, generate an image using signals corresponding to the plurality of transmit-receive pairs arranged into the aperture, and output the image to a display in communication with the processor.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1A is a diagrammatic schematic view of an intraluminal imaging system, according to aspects of the present disclosure.

FIG. 1B is a schematic diagram of a processor circuit, according to embodiments of the present disclosure.

FIG. 2 is a diagrammatic view of the top of a scanner assembly in a flat configuration, according to aspects of the present disclosure.

FIG. 3 is a diagrammatic perspective view of the scanner assembly shown in FIG. 2 in a rolled configuration around a support member, according to aspects of the present disclosure.

FIG. 4 is a diagrammatic cross-sectional side view of a scanner assembly in a rolled configuration around a support member, according to aspects of the present disclosure.

FIG. 5 is a diagrammatic graphical view of an ultrasound pulse sequence, according to aspects of the present disclosure.

FIGS. 6A and 6B are diagrammatic schematic views of an array of acoustic elements with an aperture activated to obtain image data of a target, according to aspects of the present disclosure.

FIG. 7 is a flow diagram illustrating a method for generating ultrasound images using scan sequences with accelerated aperture acquisition, according to aspects of the present disclosure.

FIG. 8A is a diagrammatic graphical view of an ultrasound pulse sequence for accelerated aperture acquisition, according to aspects of the present disclosure.

FIG. 8B is a diagrammatic graphical view of an ultrasound pulse sequence for accelerated aperture acquisition, according to aspects of the present disclosure.

FIG. 9 is a diagrammatic graphical view of an ultrasound pulse sequence for accelerated aperture acquisition, according to aspects of the present disclosure.

FIG. 10 is a diagrammatic graphical view of an ultrasound pulse sequence for accelerated aperture acquisition, according to aspects of the present disclosure.

FIG. 11A is an ultrasound image obtained using a full aperture scan sequence, according to aspects of the present disclosure.

FIG. 11B is an ultrasound image obtained using a seven-diagonal accelerated scan sequence, according to aspects of the present disclosure.

FIG. 11C is an ultrasound image obtained using a two-diagonal accelerated scan sequence, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

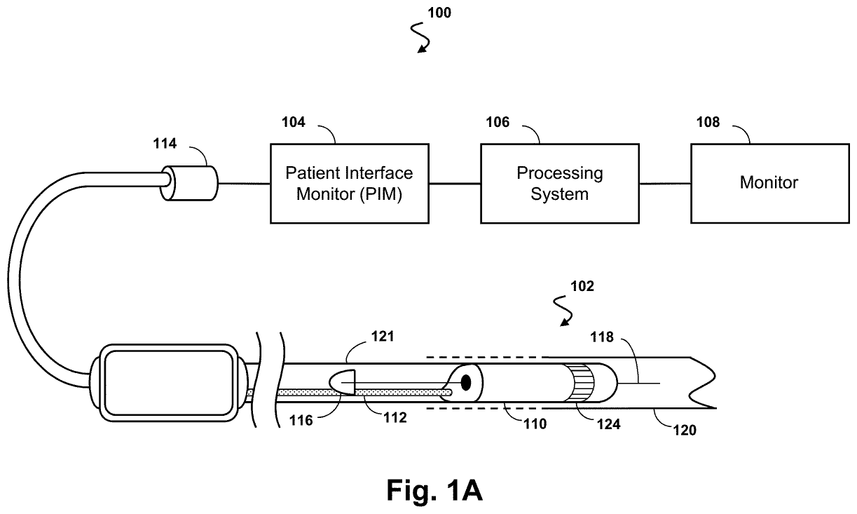

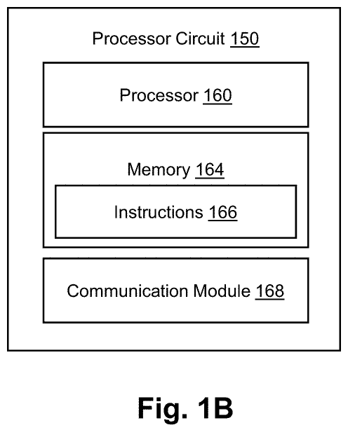

FIG. 1A is a diagrammatic schematic view of an ultrasound imaging system 100, according to aspects of the present disclosure. The ultrasound imaging system 100 can be an intraluminal imaging system. In some instances, the system 100 can be an intravascular ultrasound (IVUS) imaging system. The system 100 may include an intraluminal imaging device 102 such as a catheter, guide wire, or guide catheter, a patient interface module (PIM) 104, a processing system or console 106, and a monitor 108. The intraluminal imaging device 102 can be an ultrasound imaging device. In some instances, the device 102 can be IVUS imaging device, such as a solid-state IVUS device.

At a high level, the IVUS device 102 emits ultrasonic energy, or ultrasound signals, from a transducer array 124 included in scanner assembly 110 mounted near a distal end of the catheter device. The ultrasonic energy is reflected by tissue structures in the medium, such as a vessel 120, or another body lumen surrounding the scanner assembly 110, and the ultrasound echo signals are received by the transducer array 124. In that regard, the device 102 can be sized, shaped, or otherwise configured to be positioned within the body lumen of a patient. The PIM 104 transfers the received echo signals to the console or computer 106 where the ultrasound image (including the flow information) is reconstructed and displayed on the monitor 108. The console or computer 106 can include a processor and a memory. The computer or computing device 106 can be operable to facilitate the features of the IVUS imaging system 100 described herein. For example, the processor can execute computer readable instructions stored on the non-transitory tangible computer readable medium.

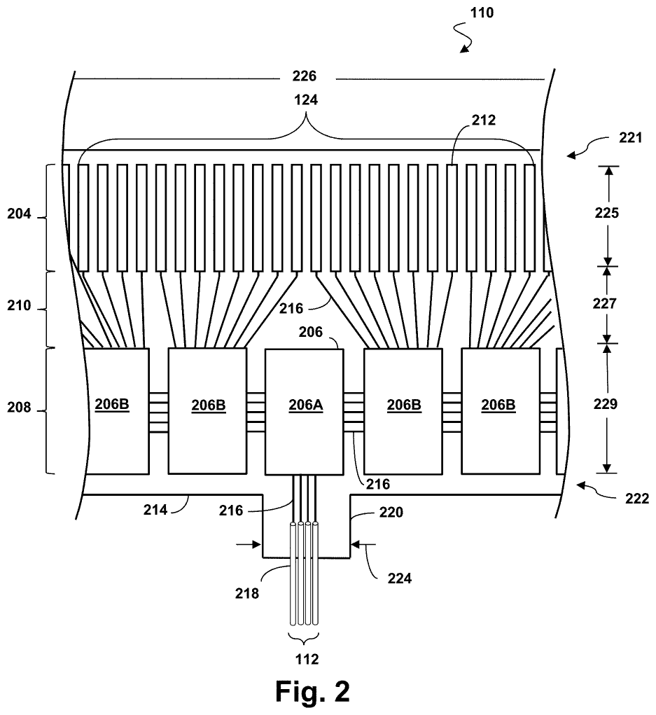

The PIM 104 facilitates communication of signals between the IVUS console 106 and the scanner assembly 110 included in the IVUS device 102. This communication includes the steps of: (1) providing commands to integrated circuit controller chip(s) 206A, 206B, illustrated in FIG. 2, included in the scanner assembly 110 to select the particular transducer array element(s), or acoustic element(s), to be used for transmit and receive, (2) providing the transmit trigger signals to the integrated circuit controller chip(s) 206A, 206B included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s) 126 of the scanner assembly 110. In some embodiments, the PIM 104 performs preliminary processing of the echo data prior to relaying the data to the console 106. In examples of such embodiments, the PIM 104 performs amplification, filtering, and/or aggregating of the data. In an embodiment, the PIM 104 also supplies high- and low-voltage DC power to support operation of the device 102 including circuitry within the scanner assembly 110.

The IVUS console 106 receives the echo data from the scanner assembly 110 by way of the PIM 104 and processes the data to reconstruct an image of the tissue structures in the medium surrounding the scanner assembly 110. The console 106 outputs image data such that an image of the vessel 120, such as a cross-sectional image of the vessel 120, is displayed on the monitor 108. Vessel 120 may represent fluid filled or surrounded structures, both natural and man-made. The vessel 120 may be within a body of a patient. The vessel 120 may be a blood vessel, as an artery or a vein of a patient's vascular system, including cardiac vasculature, peripheral vasculature, neural vasculature, renal vasculature, and/or or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be used to examine man-made structures such as, but without limitation, heart valves, stents, shunts, filters and other devices.

In some embodiments, the IVUS device includes some features similar to traditional solid-state IVUS catheters, such as the EagleEye® catheter available from Koninklijke Philips N.V. and those disclosed in U.S. Pat. No. 7,846,101 hereby incorporated by reference in its entirety. For example, the IVUS device 102 includes the scanner assembly 110 near a distal end of the device 102 and a transmission line bundle 112 extending along the longitudinal body of the device 102. The transmission line bundle or cable 112 can include a plurality of conductors, including one, two, three, four, five, six, seven, or more conductors 218 (FIG. 2). It is understood that any suitable gauge wire can be used for the conductors 218. In an embodiment, the cable 112 can include a four-conductor transmission line arrangement with, e.g., 41 AWG gauge wires. In an embodiment, the cable 112 can include a seven-conductor transmission line arrangement utilizing, e.g., 44 AWG gauge wires. In some embodiments, 43 AWG gauge wires can be used.

The transmission line bundle 112 terminates in a PIM connector 114 at a proximal end of the device 102. The PIM connector 114 electrically couples the transmission line bundle 112 to the PIM 104 and physically couples the IVUS device 102 to the PIM 104. In an embodiment, the IVUS device 102 further includes a guide wire exit port 116. Accordingly, in some instances the IVUS device is a rapid-exchange catheter. The guide wire exit port 116 allows a guide wire 118 to be inserted towards the distal end in order to direct the device 102 through the vessel 120.

In an embodiment, the image processing system 106 generates flow data by processing the echo signals from the IVUS device 102 into Doppler power or velocity information. The image processing system 106 may also generate B-mode data by applying envelope detection and logarithmic compression on the conditioned echo signals. The processing system 106 can further generate images in various views, such as 2D and/or 3D views, based on the flow data or the B-mode data. The processing system 106 can also perform various analyses and/or assessments. For example, the processing system 106 can apply virtual histology (VH) techniques, for example, to analyze or assess plaques within a vessel (e.g., the vessel 120). The images can be generated to display a reconstructed color-coded tissue map of plaque composition superimposed on a cross-sectional view of the vessel.

In an embodiment, the processing system 106 can apply a blood flow detection algorithm (e.g., ChromaFlo) to determine the movement of blood flow, for example, by acquiring image data of a target region (e.g., the vessel 120) repeatedly and determining the movement of the blood flow from the image data. The blood flow detection algorithm operates based on the principle that signals measured from vascular tissue are relatively static from acquisition to acquisition, whereas signals measured from blood flow vary at a characteristic rate corresponding to the flow rate. As such, the blood flow detection algorithm may determine movements of blood flow based on variations in signals measured from the target region between repeated acquisitions. To acquire the image data repeatedly, the processing system 106 may control to the device 102 to transmit repeated pulses on the same aperture.

While the present disclosure describes embodiments related to intravascular ultrasound (IVUS) imaging using an intravascular catheter or guidewire, it is understood that one or more aspects of the present disclosure can be implemented in any suitable ultrasound imaging system, including a synthetic aperture ultrasound imaging system, a phased array ultrasound imaging system, or any other array-based ultrasound imaging system. For example, aspects of the present disclosure can be implemented in intraluminal ultrasound imaging systems using an intracardiac (ICE) echocardiography catheter and/or a transesophageal echocardiography (TEE) probe, and/or external ultrasound imaging system using an ultrasound probe configured for imaging while positioned adjacent to and/or in contact with the patient's skin. The ultrasound imaging device can be a transthoracic echocardiography (TTE) imaging device in some embodiments.

An ultrasound transducer array of ultrasound imaging device includes an array of acoustic elements configured to emit ultrasound energy and receive echoes corresponding to the emitted ultrasound energy. In some instances, the array may include any number of ultrasound transducer elements. For example, the array can include between 2 acoustic elements and 10000 acoustic elements, including values such as 2 acoustic elements, 4 acoustic elements, acoustic elements, 64 acoustic elements, 128 acoustic elements, 500 acoustic elements, 812 acoustic elements, 3000 acoustic elements, 9000 acoustic elements, and/or other values both larger and smaller. In some instances, the transducer elements of the array may be arranged in any suitable configuration, such as a linear array, a planar array, a curved array, a curvilinear array, a circumferential array, an annular array, a phased array, a matrix array, a one-dimensional (1D) array, a 1.× dimensional array (e.g., a 1.5D array), or a two-dimensional (2D) array. The array of transducer elements (e.g., one or more rows, one or more columns, and/or one or more orientations) can be uniformly or independently controlled and activated. The array can be configured to obtain one-dimensional, two-dimensional, and/or three-dimensional images of patient anatomy.

The ultrasound transducer elements may include piezoelectric/piezoresistive elements, piezoelectric micromachined ultrasound transducer (PMUT) elements, capacitive micromachined ultrasound transducer (CMUT) elements, and/or any other suitable type of ultrasound transducer elements. The ultrasound transducer elements of the array are in communication with (e.g., electrically coupled to) electronic circuitry. For example, the electronic circuitry can include one or more transducer control logic dies. The electronic circuitry can include one or more integrated circuits (IC), such as application specific integrated circuits (ASICs). In some embodiments, one or more of the ICs can include a microbeamformer (OF). In other embodiments, one or more of the ICs includes a multiplexer circuit (MUX).

FIG. 1B is a schematic diagram of a processor circuit 150, according to embodiments of the present disclosure. The processor circuit 150 may be implemented in the processing system 106 and/or the imaging device 102 of FIG. 1A. As shown, the processor circuit 150 may include a processor 160, a memory 164, and a communication module 168. These elements may be in direct or indirect communication with each other, for example via one or more buses.

The processor 160 may include a central processing unit (CPU), a digital signal processor (DSP), an ASIC, a controller, an FPGA, another hardware device, a firmware device, or any combination thereof configured to perform the operations described herein. The processor 160 may also be implemented as a combination of computing devices, e.g., a combination of a DSP and a microprocessor, a plurality of microprocessors, one or more microprocessors in conjunction with a DSP core, or any other such configuration.

The memory 164 may include a cache memory (e.g., a cache memory of the processor 160), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 164 includes a non-transitory computer-readable medium. The memory 164 may store instructions 166. The instructions 166 may include instructions that, when executed by the processor 160, cause the processor 160 to perform the operations described herein with reference to the processing system 106 and/or the imaging device 102 (FIG. 1A). Instructions 166 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 168 can include any electronic circuitry and/or logic circuitry to facilitate direct or indirect communication of data between the processor circuit 150, the imaging device 102, and/or the display 108. In that regard, the communication module 168 can be an input/output (I/O) device. In some instances, the communication module 168 facilitates direct or indirect communication between various elements of the processor circuit 150 and/or the processing system 106 (FIG. 1A).

FIG. 2 is a diagrammatic top view of a portion of a flexible assembly 200, according to aspects of the present disclosure. The flexible assembly 200 includes a transducer array 124 formed in a transducer region 204 and transducer control logic dies 206 (including dies 206A and 206B) formed in a control region 208, with a transition region 210 disposed therebetween.

The transducer control logic dies 206 are mounted on a flexible substrate 214 into which the transducers 212 have been previously integrated. The flexible substrate 214 is shown in a flat configuration in FIG. 2. Though six control logic dies 206 are shown in FIG. 2, any number of control logic dies 206 may be used. For example, one, two, three, four, five, six, seven, eight, nine, ten, or more control logic dies 206 may be used.

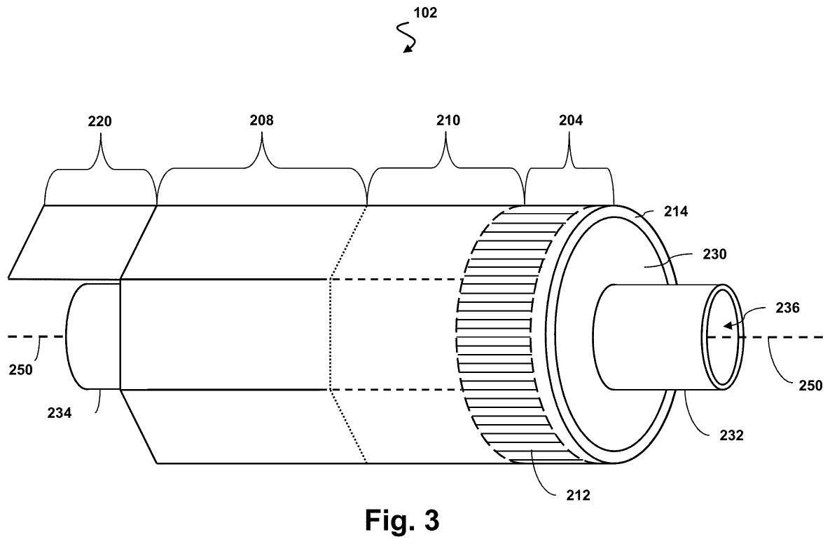

The flexible substrate 214, on which the transducer control logic dies 206 and the transducers 212 are mounted, provides structural support and interconnects for electrical coupling. The flexible substrate 214 may be constructed to include a film layer of a flexible polyimide material such as KAPTON™ (trademark of DuPont). Other suitable materials include polyester films, polyimide films, polyethylene napthalate films, or polyetherimide films, liquid crystal polymer, other flexible printed semiconductor substrates as well as products such as Upilex® (registered trademark of Ube Industries) and TEFLON® (registered trademark of E.I. du Pont). In the flat configuration illustrated in FIG. 2, the flexible substrate 214 has a generally rectangular shape. As shown and described herein, the flexible substrate 214 is configured to be wrapped around a support member 230 (FIG. 3) in some instances. Therefore, the thickness of the film layer of the flexible substrate 214 is generally related to the degree of curvature in the final assembled flexible assembly 110. In some embodiments, the film layer is between 5 μm and 100 μm, with some particular embodiments being between 5 μm and 25.1 μm, e.g., 6 μm.

The transducer control logic dies 206 is a non-limiting example of a control circuit. The transducer region 204 is disposed at a distal portion 221 of the flexible substrate 214. The control region 208 is disposed at a proximal portion 222 of the flexible substrate 214. The transition region 210 is disposed between the control region 208 and the transducer region 204. Dimensions of the transducer region 204, the control region 208, and the transition region 210 (e.g., lengths 225, 227, 229) can vary in different embodiments. In some embodiments, the lengths 225, 227, 229 can be substantially similar or, the length 227 of the transition region 210 may be less than lengths 225 and 229, the length 227 of the transition region 210 can be greater than lengths 225, 229 of the transducer region and controller region, respectively.

The control logic dies 206 are not necessarily homogenous. In some embodiments, a single controller is designated a master control logic die 206A and contains the communication interface for cable 142 which may serve as an electrical conductor, e.g., electrical conductor 112, between a processing system, e.g., processing system 106, and the flexible assembly 200. Accordingly, the master control circuit may include control logic that decodes control signals received over the cable 142, transmits control responses over the cable 142, amplifies echo signals, and/or transmits the echo signals over the cable 142. The remaining controllers are slave controllers 206B. The slave controllers 206B may include control logic that drives a transducer 212 to emit an ultrasonic signal and selects a transducer 212 to receive an echo. In the depicted embodiment, the master controller 206A does not directly control any transducers 212. In other embodiments, the master controller 206A drives the same number of transducers 212 as the slave controllers 206B or drives a reduced set of transducers 212 as compared to the slave controllers 206B. In an exemplary embodiment, a single master controller 206A and eight slave controllers 206B are provided with eight transducers assigned to each slave controller 206B.

To electrically interconnect the control logic dies 206 and the transducers 212, in an embodiment, the flexible substrate 214 includes conductive traces 216 formed in the film layer that carry signals between the control logic dies 206 and the transducers 212. In particular, the conductive traces 216 providing communication between the control logic dies 206 and the transducers 212 extend along the flexible substrate 214 within the transition region 210. In some instances, the conductive traces 216 can also facilitate electrical communication between the master controller 206A and the slave controllers 206B. The conductive traces 216 can also provide a set of conductive pads that contact the conductors 218 of cable 142 when the conductors 218 of the cable 142 are mechanically and electrically coupled to the flexible substrate 214. Suitable materials for the conductive traces 216 include copper, gold, aluminum, silver, tantalum, nickel, and tin, and may be deposited on the flexible substrate 214 by processes such as sputtering, plating, and etching. In an embodiment, the flexible substrate 214 includes a chromium adhesion layer. The width and thickness of the conductive traces 216 are selected to provide proper conductivity and resilience when the flexible substrate 214 is rolled. In that regard, an exemplary range for the thickness of a conductive trace 216 and/or conductive pad is between 1-5 μm. For example, in an embodiment, 5 μm conductive traces 216 are separated by 5 μm of space. The width of a conductive trace 216 on the flexible substrate may be further determined by the width of the conductor 218 to be coupled to the trace/pad.

The flexible substrate 214 can include a conductor interface 220 in some embodiments. The conductor interface 220 can be a location of the flexible substrate 214 where the conductors 218 of the cable 142 are coupled to the flexible substrate 214. For example, the bare conductors of the cable 142 are electrically coupled to the flexible substrate 214 at the conductor interface 220. The conductor interface 220 can be tab extending from the main body of flexible substrate 214. In that regard, the main body of the flexible substrate 214 can refer collectively to the transducer region 204, controller region 208, and the transition region 210. In the illustrated embodiment, the conductor interface 220 extends from the proximal portion 222 of the flexible substrate 214. In other embodiments, the conductor interface 220 is positioned at other parts of the flexible substrate 214, such as the distal portion 221, or the flexible substrate 214 may lack the conductor interface 220. A value of a dimension of the tab or conductor interface 220, such as a width 224, can be less than the value of a dimension of the main body of the flexible substrate 214, such as a width 226. In some embodiments, the substrate forming the conductor interface 220 is made of the same material(s) and/or is similarly flexible as the flexible substrate 214. In other embodiments, the conductor interface 220 is made of different materials and/or is comparatively more rigid than the flexible substrate 214. For example, the conductor interface 220 can be made of a plastic, thermoplastic, polymer, hard polymer, etc., including polyoxymethylene (e.g., DELRIN®), polyether ether ketone (PEEK), nylon, Liquid Crystal Polymer (LCP), and/or other suitable materials.

FIG. 3 illustrates a perspective view of the device 102 with the scanner assembly 110 in a rolled configuration. In some instances, the assembly 110 is transitioned from a flat configuration (FIG. 2) to a rolled or more cylindrical configuration (FIG. 3). For example, in some embodiments, techniques are utilized as disclosed in one or more of U.S. Pat. No. 6,776,763, titled “ULTRASONIC TRANSDUCER ARRAY AND METHOD OF MANUFACTURING THE SAME” and U.S. Pat. No. 7,226,417, titled “HIGH RESOLUTION INTRAVASCULAR ULTRASOUND SENSING ASSEMBLY HAVING A FLEXIBLE SUBSTRATE,” each of which is hereby incorporated by reference in its entirety.

In some embodiments, the transducer elements 212 and/or the controllers 206 can be positioned in in an annular configuration, such as a circular configuration or in a polygon configuration, around a longitudinal axis 250 of a support member 230. It will be understood that the longitudinal axis 250 of the support member 230 may also be referred to as the longitudinal axis of the scanner assembly 110, the flexible elongate member 121, and/or the device 102. For example, a cross-sectional profile of the imaging assembly 110 at the transducer elements 212 and/or the controllers 206 can be a circle or a polygon. Any suitable annular polygon shape can be implemented, such as a based on the number of controllers/transducers, flexibility of the controllers/transducers, etc., including a pentagon, hexagon, heptagon, octagon, nonagon, decagon, etc. In some examples, the plurality of transducer controllers 206 may be used for controlling the plurality of ultrasound transducer elements 212 to obtain imaging data associated with the vessel 120.

The support member 230 can be referenced as a unibody in some instances. The support member 230 can be composed of a metallic material, such as stainless steel, or non-metallic material, such as a plastic or polymer as described in U.S. Provisional Application No. 61/985,220, “Pre-Doped Solid Substrate for Intravascular Devices,” filed Apr. 28, 2014, ('220 Application) the entirety of which is hereby incorporated by reference herein. The support member 230 can be a ferrule having a distal flange or portion 232 and a proximal flange or portion 234. The support member 230 can be tubular in shape and define a lumen 236 extending longitudinally therethrough. The lumen 236 can be sized and shaped to receive the guide wire 118. The support member 230 can be manufactured using any suitable process. For example, the support member 230 can be machined and/or electrochemically machined or laser milled, such as by removing material from a blank to shape the support member 230, or molded, such as by an injection molding process.

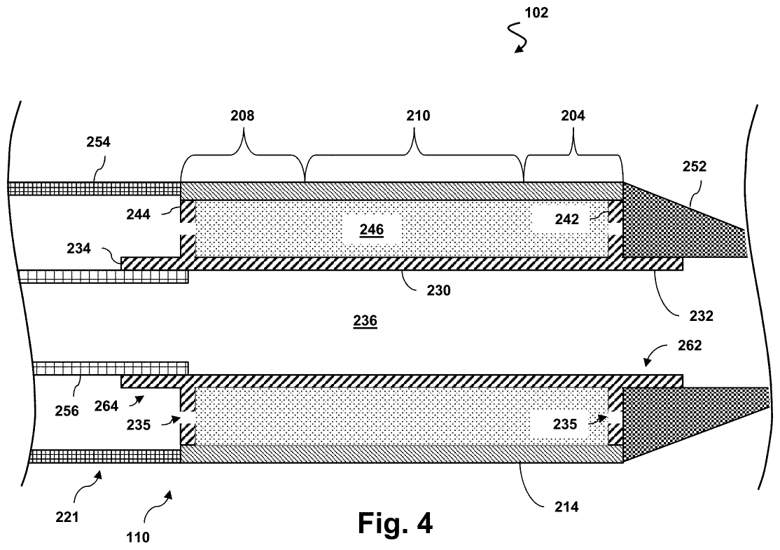

Referring now to FIG. 4, shown there is a diagrammatic cross-sectional side view of a distal portion of the intraluminal imaging device 102, including the flexible substrate 214 and the support member 230, according to aspects of the present disclosure. The support member 230 can be referenced as a unibody in some instances. The support member 230 can be composed of a metallic material, such as stainless steel, or non-metallic material, such as a plastic or polymer as described in U.S. Provisional Application No. 61/985,220, “Pre-Doped Solid Substrate for Intravascular Devices,” filed Apr. 28, 2014, the entirety of which is hereby incorporated by reference herein. The support member 230 can be ferrule having a distal portion 262 and a proximal portion 264. The support member 230 can define a lumen 236 extending along the longitudinal axis LA. The lumen 236 is in communication with the entry/exit port 116 and is sized and shaped to receive the guide wire 118 (FIG. 1A). The support member 230 can be manufactured according to any suitable process. For example, the support member 230 can be machined and/or electrochemically machined or laser milled, such as by removing material from a blank to shape the support member 230, or molded, such as by an injection molding process. In some embodiments, the support member 230 may be integrally formed as a unitary structure, while in other embodiments the support member 230 may be formed of different components, such as a ferrule and stands 242, 244, that are fixedly coupled to one another. In some cases, the support member 230 and/or one or more components thereof may be completely integrated with inner member 256. In some cases, the inner member 256 and the support member 230 may be joined as one, e.g., in the case of a polymer support member.

Stands 242, 244 that extend vertically are provided at the distal and proximal portions 262, 264, respectively, of the support member 230. The stands 242, 244 elevate and support the distal and proximal portions of the flexible substrate 214. In that regard, portions of the flexible substrate 214, such as the transducer portion or region 204, can be spaced from a central body portion of the support member 230 extending between the stands 242, 244. The stands 242, 244 can have the same outer diameter or different outer diameters. For example, the distal stand 242 can have a larger or smaller outer diameter than the proximal stand 244 and can also have special features for rotational alignment as well as control chip placement and connection. To improve acoustic performance, any cavities between the flexible substrate 214 and the surface of the support member 230 are filled with a backing material 246. The liquid backing material 246 can be introduced between the flexible substrate 214 and the support member 230 via passageways 235 in the stands 242, 244. In some embodiments, suction can be applied via the passageways 235 of one of the stands 242, 244, while the liquid backing material 246 is fed between the flexible substrate 214 and the support member 230 via the passageways 235 of the other of the stands 242, 244. The backing material can be cured to allow it to solidify and set. In various embodiments, the support member 230 includes more than two stands 242, 244, only one of the stands 242, 244, or neither of the stands. In that regard the support member 230 can have an increased diameter distal portion 262 and/or increased diameter proximal portion 264 that is sized and shaped to elevate and support the distal and/or proximal portions of the flexible substrate 214.

The support member 230 can be substantially cylindrical in some embodiments. Other shapes of the support member 230 are also contemplated including geometrical, non-geometrical, symmetrical, non-symmetrical, cross-sectional profiles. As the term is used herein, the shape of the support member 230 may reference a cross-sectional profile of the support member 230. Different portions the support member 230 can be variously shaped in other embodiments. For example, the proximal portion 264 can have a larger outer diameter than the outer diameters of the distal portion 262 or a central portion extending between the distal and proximal portions 262, 264. In some embodiments, an inner diameter of the support member 230 (e.g., the diameter of the lumen 236) can correspondingly increase or decrease as the outer diameter changes. In other embodiments, the inner diameter of the support member 230 remains the same despite variations in the outer diameter.

A proximal inner member 256 and a proximal outer member 254 are coupled to the proximal portion 264 of the support member 230. The proximal inner member 256 and/or the proximal outer member 254 can include a flexible elongate member. The proximal inner member 256 can be received within a proximal flange 234. The proximal outer member 254 abuts and is in contact with the flexible substrate 214. A distal member 252 is coupled to the distal portion 262 of the support member 230. For example, the distal member 252 is positioned around the distal flange 232. The distal member 252 can abut and be in contact with the flexible substrate 214 and the stand 242. The distal member 252 can be the distal-most component of the intraluminal imaging device 102.

One or more adhesives can be disposed between various components at the distal portion of the intraluminal imaging device 102. For example, one or more of the flexible substrate 214, the support member 230, the distal member 252, the proximal inner member 256, and/or the proximal outer member 254 can be coupled to one another via an adhesive.

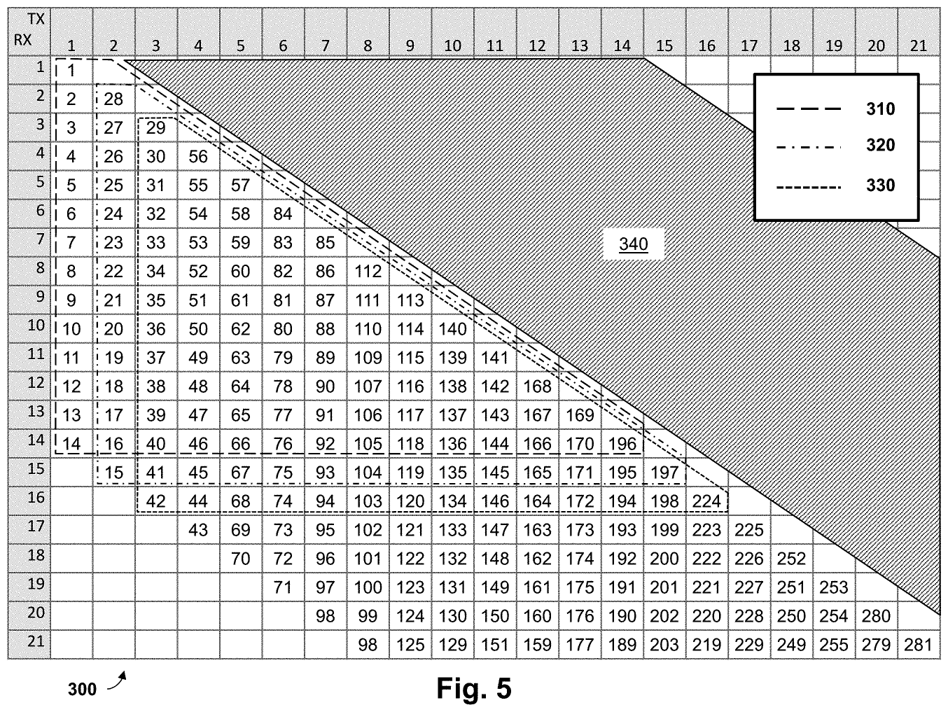

The assembly 110 shown in FIG. 2 can be activated according to a pulse sequence or scan sequence to form coherent beams of ultrasound energy to generate an image. In that regard, FIG. 5 is a diagrammatic graphical view showing an ultrasound pulse sequence of a solid-state IVUS device. The pulse sequence 300 includes a contiguous “zig-zag” pattern or arrangement of transmit-receive pairs, which can alternatively be described as transmit-receive events. Each transmit-receive pair is represented by an index, or number, corresponding to a sequential time at which the corresponding transmit-receive pair is activated to obtain ultrasound imaging data. In that regard, each transmit-receive index is an integer representing its relative temporal position in the sequence 300. In the embodiment of FIG. 5, each transmit-receive index corresponds to a single transmit-receive pair. Each transmit-receive pair is defined by a transmit element index, shown on the x-axis, and a receive element index, shown on the y-axis. Each transmit element index and receive element index corresponds to an ultrasound element of an array of ultrasound transducer elements. In the embodiment shown in FIG. 5, the array includes 64 ultrasound transducer elements. It may be advantageous in some circumstances, for instance to improve sensitivity or limit element directivity, to transmit or receive on more than one element at a time following a similar arrangement to that described above and illustrated in FIG. 5. The term “transmit element” as used here and throughout this document may therefore be taken to represent one or more physical elements transmitting together and associated with a nominal transmit element location within the array. Similarly, the term “receive element” as used here and throughout this document, may therefore be taken to represent one or more physical elements connected together with a nominal receive element location within the array.

For example, the transmit-receive pair associated with transmit-receive index “1” is defined by transmit element index number 1 and receive element index 1. In some embodiments, the transmit element index and receive element index correspond to the same ultrasound transducer element. In other embodiments, the transmit element index and receive element index correspond to different ultrasound transducer elements. For example, the transmit-receive pair numbered “2,” which is shown directly below transmit-receive pair 1, is defined by transmit element index 1 and receive element index 2. That is, the ultrasound imaging data associated with transmit-receive pair 2 is obtained by activating transmit element index 1 to transmit ultrasound energy into the patient volume, and then activating receive element index 2 to receive ultrasound echoes from the patient volume. In FIG. 5, 294 transmit-receive pairs of an ultrasound pulse sequence are shown. Each transmit-receive pair is activated sequentially according to its transmit-receive index.

In the sequence 300, the ultrasound transducer element associated with transmit index 1 transmits fourteen consecutive times, while the elements associated with receive indices 1 through 14 are sequentially activated to receive the corresponding echoes. Next, the element associated with transmit index 2 transmits fourteen consecutive times, while the elements associated with receive indices 15 through 2 (stepping backward) are sequentially activated to receive the corresponding echoes. This sequence continues in a zig-zag pattern around the array of ultrasound transducer elements. Each transmit-receive pair is associated with one or more apertures 310, 320, 330. The apertures 310, 320, 330 are associated with different sections, angular portions, or arcs of the array. For example, a first aperture 310 includes transmit-receive pairs spanning from index 1 to index 196, a second aperture 320 includes transmit-receive pairs spanning from index 15 to index 197, and a third aperture 330 includes transmit-receive pairs spanning from index 29 to index 224. The transmit-receive pairs in each aperture are combined to form an A-line for a B-mode image. Thus, the transmit-receive pairs contained within the first aperture 310 are combined to form a first A-line, the transmit-receive pairs contained within the second aperture 320 are combined to form a second A-line, the transmit-receive pairs contained within the third aperture are combined to form a third A-line, and so on. The A-line formed by the first aperture 310 will be centered between transmit and receive element indices 7 and 8, the A-line formed by the second aperture 320 will be centered between transmit and receive element indices numbered 8 and 9, the A-line formed by the third aperture 330 will be centered between transmit and receive element indices numbered 9 and 10, and so on. Several apertures are used to form A-lines, which are combined and arranged to form a B-mode image.

A non-fired region 340 indicates complementary transmit-receive pairs that are not fired due to the reciprocity rule. In that regard, given an aperture of N elements, a naive approach to synthetic aperture ultrasound imaging would be to acquire all N\hat{ }2 possible transmit/receive combinations in the aperture (i.e. transmit on element i, receive on element j for all i and j less than or equal to N). In order to remove redundant information and firings from an acquisition sequences to streamline acquisition, each pair of transmit and receive elements are sampled or fired a single time, while the complementary pair of receive and transmit elements are skipped or not fired. Thus, if a scan sequence transmits on element i and receives on element j, then the scan sequence may not also transmit on j and receive on i. Accordingly, many transmit-receive pairs can be skipped or omitted from a synthetic aperture acquisition sequence. Instead of requiring N\hat{ }2 acquisitions, reciprocity allows us to acquire the same information with only N(N+1)/2 acquisitions. This approach can be referred to as the reciprocity rule. For example, in FIG. 5, the sequence 300 includes transmit-receive index 5, corresponding to transmit element 1 and receive element 5. Accordingly, the complementary transmit-receive pair associated with transmit element 5 and receive element 1, which theoretically comprises the same signal characteristics (e.g., spatial frequency, signal-to-noise ratio (SNR)), is not activated in the sequence 300. The non-fired region 340 illustrates the group of non-fired, complementary transmit-receive pairs corresponding to the fired transmit-receive pairs of indices 1-281.

It will be understood that the scan sequence shown in FIG. 5 is exemplary and that other scan sequences can be used besides the sequence shown in FIG. 5. For example, the present disclosure contemplates scan sequences using patterns of transmit-receive pairs, aperture sizes, and combinations of transmit and receive pairs that are different from those described above.

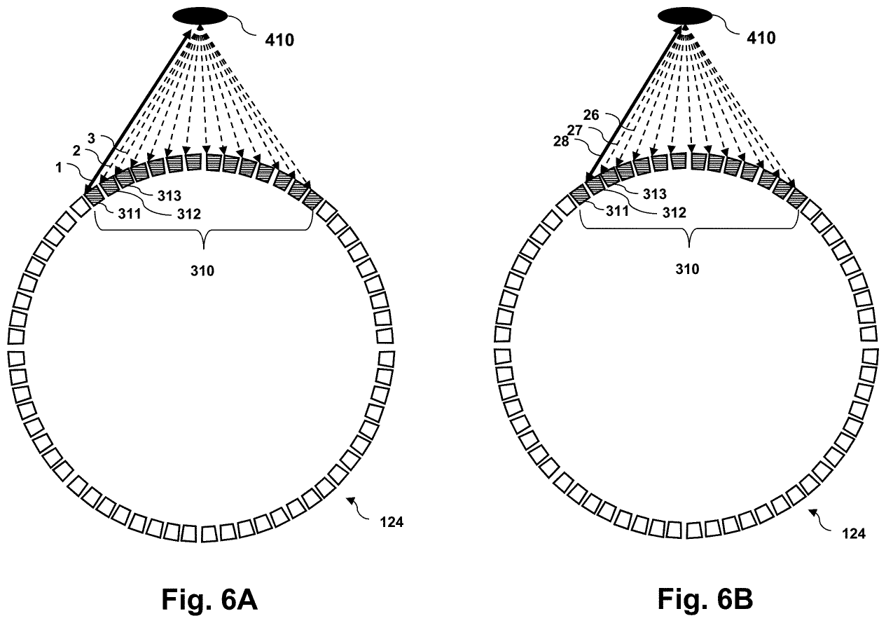

FIGS. 6A and 6B depict an annular array of an IVUS imaging device performing a portion of the scan sequence 300 shown in FIG. 5. In particular, FIGS. 6A and 6B show a portion of the scan sequence 300 associated with the first aperture 310. The first aperture 310 spans fourteen elements of the array 124. In the scan sequence 300, the first aperture 310 includes a plurality of transmit-receive pairs or firings, which are characterized by a transmit element index and a receive element index. In FIG. 6A, a first element 311 is the transmitting element, and each of the elements of the aperture 310, including the first element 311, sequentially receive ultrasound energy transmitted by the first element 311 and reflected off an object 410. In that regard, transmit-receive pair 1 is illustrated by the bold arrow in which the first element 311 is both the transmitting element and the receiving element. Transmit-receive pair 2 is characterized by ultrasound energy transmitted by the first element 311 and received by a second element 312. Transmit-receive pair 3 is characterized by ultrasound energy transmitted by the first element 311 and received by a third element 313. This transmit-receive sequence is repeated so that all fourteen elements of the aperture 310 have received ultrasound energy transmitted by the first element 311.

In FIG. 6B, the scan sequence continues with the second element 312 transmitting, and each of the remaining elements sequentially receiving the ultrasound energy transmitted by the second element 312, as similarly described above. It will be understood that, as shown in FIG. 6B, the first element 311 does not receive ultrasound energy transmitted by the second element 312. As explained above, the first element 311 may not receive ultrasound energy transmitted by the second element 312 due to the signal reciprocity rule.

The frame rate achievable by a synthetic aperture ultrasound device may be restricted by the number of transmit-receive events in the scan sequence. In some aspects, a physician may prefer to produce an ultrasound image stream at a higher frame rate without significant reductions in image quality. In other instances, a physician may be willing to trade a reduction in scan redundancy (i.e. the number of transmit-receive events), and therefore some reduction in SNR for increases in frame rate. As explained above, by using the reciprocity rule, some redundant transmit-receive pair can be used to skip or omit a plurality of transmit-receive pairs from a scan sequence. The present disclosure describes scan sequences in which additional transmit-receive pairs can be omitted in a scan sequence while sampling the same number of spatial frequencies. For example, because transmit receive pairs [i,j] and [k,l] may correspond to a same spatial frequency and therefore provide redundant information, transmit-receive pair [k,l] may be omitted. If spatial frequency of a pair of elements i and j (not necessarily distinct) in an aperture of N elements is defined as i+j−1, it follows that an aperture contains only 2N−1 distinct spatial frequencies (from 1 to 2N−1). It may be desirable that an aperture includes transmit-receive pairs that sample all 2N−1 distinct spatial frequencies. Accordingly, the present disclosure describes scan sequences, and associated devices and methods, in which fewer than N(N+1)/2 transmit/receive pairs are acquired to form the image from an N element aperture and where all 2N−1 spatial frequencies occur at least once in the acquisition sequence for the aperture.

In an exemplary embodiment, a scan sequence would include transmit-receive pairs [i,j] in which the absolute value of (i−j) is relatively small. This is due to two factors: element directivity and reuse. The further separated two elements are on the aperture, the lower the sensitivity (SNR) of the resulting echoes, since the elements are directional. Further, since we are translating our aperture slowly around the array, we can re-use transmit/receive pairs for multiple apertures. the smaller the distance between two elements, the more apertures over which they can be re-used.

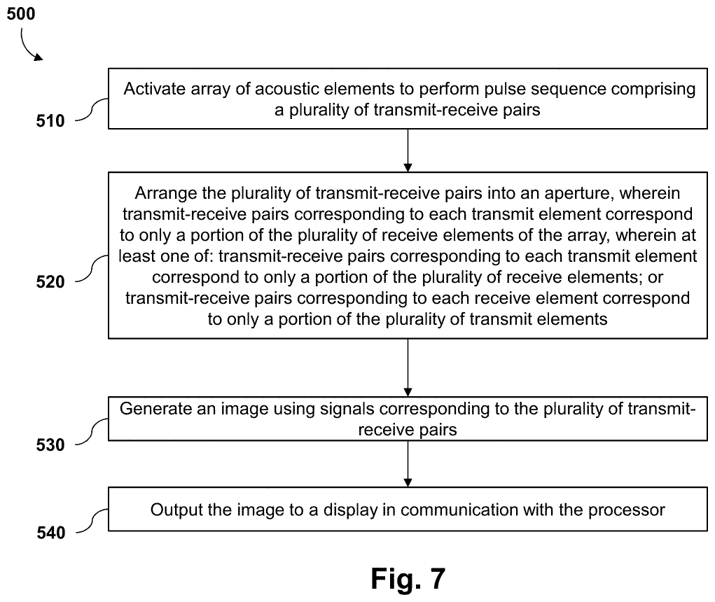

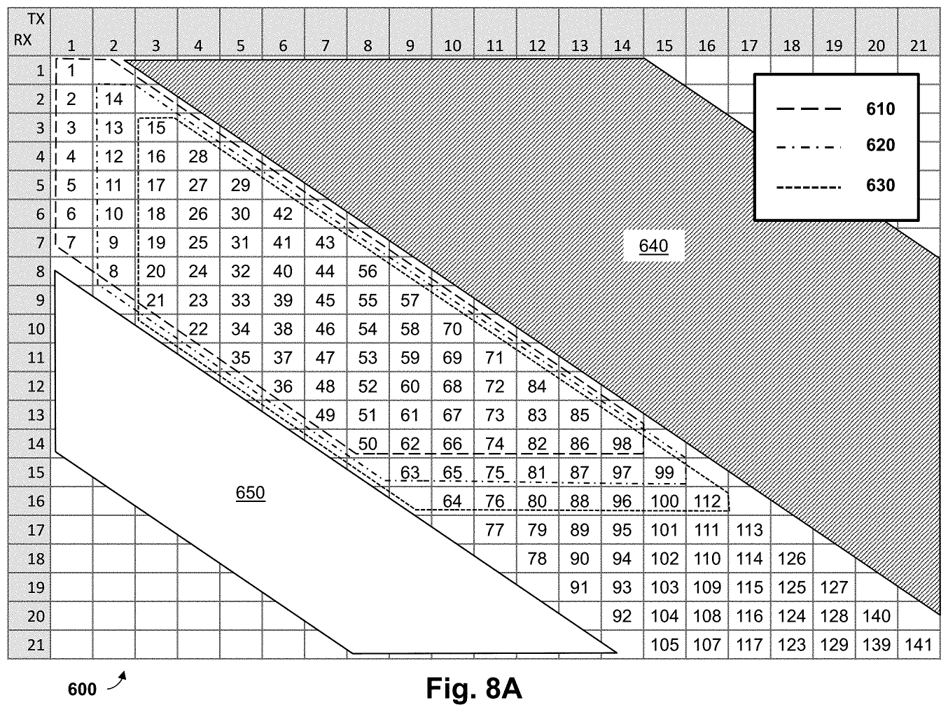

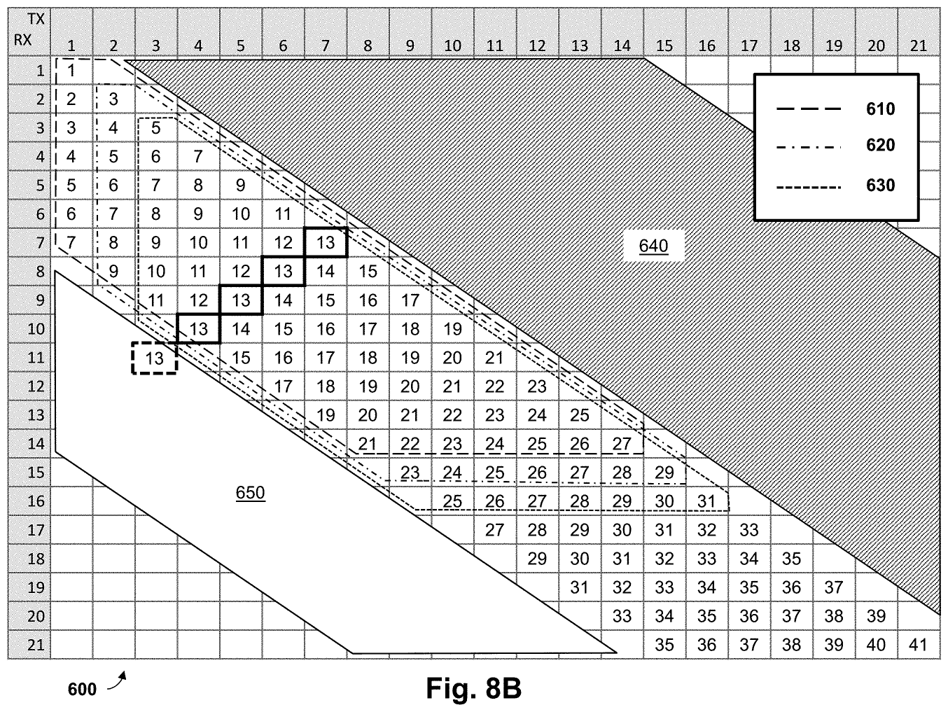

FIG. 7 illustrates a method 500 for performing a scan sequence with a controllable frame rate, according to an embodiment of the present disclosure. Aspects of the method FIG. 7 are also illustrated in FIGS. 8-10, as described below.

In step 510, a processor or controller activates an array of acoustic elements to perform a pulse or scan sequence, such as the scan sequence 300 shown in FIG. 5. In some embodiments, the array may be an annular array of an IVUS imaging catheter, such as the array 124 shown in FIGS. 6A and 6B. However, other types of arrays may also be used, such as external ultrasound imaging probes, intracardiac echocardiography (ICE) catheters, transesophageal echocardiography (TEE) probes, transthoracic echocardiography (TTE) probes, or any other suitable array-based ultrasound imaging device. Further, while exemplary embodiments of the present disclosure may describe one-dimensional arrays used to generate two-dimensional images, other types of arrays can be used, such as 1.5D arrays, 1.XD arrays, or 2D arrays for generating three-dimensional images.

The scan sequence may be performed by activating apertures of the array to form focused beams of ultrasound energy. In an exemplary embodiment, the scan sequence includes activating a sequence of transmit-receive pairs that scans in a reciprocating pattern around or across the array until every element of the array has been fired. Groups of transmit-receive pairs associated with a respective group of (e.g., fourteen) contiguous acoustic elements are arranged into apertures. In other embodiments (e.g., external ultrasound probe), multiple elements are simultaneously activated to transmit and/or receive ultrasound energy.

In step 520, the processor arranges the plurality of transmit-receive pairs into an aperture. It will be understood that the step of arranging transmit-receive pairs into an aperture may refer to selectively combining signals associated with spatially-corresponding transmit-receive pairs to generate lines of image data. In some aspects, in an aperture comprising or spanning N transmit elements and N receive elements, where the transmit and receive elements may correspond to the same acoustic elements of the array, one or more of the apertures can comprise fewer than half of N(N+1) transmit-receive pairs. Thus, the one or more transmit-receive pairs in the aperture, as well as their complementary transmit-receive pairs are not fired. However, in an exemplary embodiment, all spatial frequencies represented in the aperture are sampled. The transmit-receive pairs corresponding to each transmit element in the aperture correspond to only a portion of the plurality of receive elements of the aperture. For example, in an aperture spanning fourteen transmit elements and fourteen receive elements, the transmit-receive pairs of the aperture that are associated with any one of the fourteen transmit elements are associated with less than fourteen receive elements of the aperture. An example of the scan sequence arranged into apertures according to step 520 is shown in FIGS. 8A and 8B. In that regard, FIG. 8A shows the sequence 600 with sequential transmit-receive indices, and FIG. 8B shows the sequence 600 with the spatial frequencies corresponding to the transmit-receive pairs. The scan sequence 600 includes three apertures 610, 620, 630 comprising different but overlapping groups of transmit-receive pairs associated with different but overlapping groups of transmit elements and receive elements. A first aperture 610 comprises transmit-receive pairs associated with transmit elements 1-14 and receive elements 1-14.

Transmit-receive pairs associated with each and any given transmit element in the aperture 610 are associated with only a portion of the receive elements in the aperture 610. For example, the transmit-receive pairs associated with transmit element 1 are associated with receive elements 1-7, and not receive elements 1-14. Conversely, transmit-receive pairs associated with each and any given receive element in the aperture 610 are associated with only a portion of the transmit elements in the aperture 610. For example, in the first aperture 610, transmit-receive pairs associated with receive element 14 are associated with transmit elements 8-14, and not transmit elements 1-14. The sequence 600 can be described as a “seven-diagonal” sequence, referring to the number of transmit-receive pairs associated with any given transmit element. Accordingly, in contrast to the sequence 300 shown in FIG. 5, each aperture 610, 620, 630 of the scan sequence 600 comprises a non-triangular grouping or arrangement of transmit-receive pairs, such as a trapezoidal grouping of transmit-receive pairs. In that regard, the scan sequence 600 includes a second group 650 of non-fired or skipped transmit-receive pairs. In some embodiments, transmit-receive pairs corresponding to each transmit element correspond to only a portion of the plurality of receive elements while transmit-receive pairs corresponding to each receive element correspond to all of the transmit elements in the aperture. In other embodiments, transmit-receive pairs corresponding to each receive element correspond to only a portion of the plurality of transmit elements while transmit-receive pairs corresponding to each transmit element correspond to all of the transmit elements in the aperture. In other embodiments, transmit-receive pairs corresponding to each transmit element correspond to only a portion of the plurality of receive elements and transmit-receive pairs corresponding to each receive element correspond to only a portion of the plurality of transmit elements.

As shown in FIG. 8B, many of the transmit-receive pairs in the sequence 600 have the same spatial frequency. The spatial frequency is representative of a location of a given transmit-receive pair on the array. For example, a transmit-receive pair associated with transmit element 1 and receive element 7 has a spatial frequency of 7. This is the same as the spatial frequency of a transmit-receive pair associated with transmit element 2 and receive element 6. The spatial frequency of each transmit-receive pair in the sequence 600 is determined by adding the transmit element index and the receive element index and subtracting the added value by one. However, other relationships can be used to represent spatial frequency.

As shown in FIG. 8B, transmit-receive pairs associated with a given spatial frequency (e.g., 13) are arranged diagonally. It will be understood that the non-fired region 650 encompasses a group of non-fired transmit-receive pairs whose spatial frequencies are represented in the fired transmit-receive pairs of the sequence 600. For example, while the transmit-receive pair associated with transmit element 3 and receive element 11 is in the non-fired region 650, and therefore not fired, the spatial frequency of the transmit-receive pair is (3+11−1)=13, which is a spatial frequency represented in multiple transmit-receive pairs of the fired apertures of the sequence 600. Accordingly, while the reciprocity region 640 comprises complementary transmit-receive pairs that are not fired due to the reciprocity rule, the non-fired region 650 includes transmit-receive pairs that are not fired but correspond to spatial frequencies of one or more fired transmit-receive pairs. Thus, neither the transmit-receive pairs of the non-fired region 650, nor their complementary transmit-receive pairs in the reciprocity region 640, are fired. In the scan sequence 600 of FIGS. 8A and 8B, less than half of the potential transmit-receive pairs associated with an aperture spanning fourteen transmit elements and fourteen receive elements are fired.

Furthermore, the non-fired region 650 may comprise transmit-receive pairs that would have produced signals with lower SNR than the fired transmit-receive pairs. For example, the non-fired region 650 may comprise transmit-receive pairs where the absolute value of (i−j) is less than K for receive apertures larger than K. Accordingly, while the sequence 600 shown in FIGS. 8A and 8B includes fewer transmit-receive pairs compared to the sequence 300 of FIG. 5, the sequence 600 of FIGS. 8A and 8B is activated using the same range of spatial frequencies as the sequence 300, but with the lower SNR transmit-receive pairs skipped or not fired. By skipping or omitting the lower SNR transmit-receive pairs, the scan sequence 600 can be performed in less time while potential degradation of the resulting image is mitigated.

In some embodiments, step 520 can include combining signals associated with the plurality of transmit-receive pairs using beamforming. Further, in some embodiments, step 520 includes applying an Apodization scheme to the signals to improve SNR of the combined signal. For example, more weight could be applied to transmit-receive pairs in which the transmit and receive elements are close to each other, and less weight transmit-receive pairs in which the transmit and receive elements are far from each other. In that regard, an Apodization scheme can be applied using the relationship:

$${w}_{round \text{-} trip} ( k ) = {\sum }_{tx + rv = k} w ( t x ) w ( r v )$$

where w(i) is the Apodization weight for a given transmit or receive element i, and (tx+rx)=k represents a given transmit-receive pair. For a scan sequence of M diagonals and an aperture spanning N transmit and receive elements, it may be desirable to normalize the signals of each transmit-receive pair before summing or beamforming. In some embodiments, the normalization is performed using the relationship below:

$${α}_{normalize} ( k ) = \frac{{\sum }_{tx + rv = k , \vert tx - rv \vert < N} w ( tx ) w ( rv )}{{\sum }_{tx + rv = k , \vert tx - rv \vert < M} w ( tx ) w ( rv )}$$

By normalizing the transmit-receive pairs, the point spread function (psf) of each aperture can be maintained similar to full aperture scan sequences. Further, in some embodiments, a matched filter round-trip Apodization can be applied that further attenuates transmit-receive pairs in which the transmit and receive elements are spaced far from one another. The matched filter Apodization can be applied based on element directivity and depth of the signal.

In step 530, the processor generates an image using the signals corresponding to the plurality of transmit-receive pairs of the aperture. In step 540, the image is output to a display in communication with the processor. In practice, the techniques described above are applied to a plurality of apertures, with the signals of each aperture combined to generate a single line of image data. The lines of image data are then combined into an ultrasound image. Because some transmit-receive pairs in the sequence 600 are skipped or omitted, the time to perform the sequence can be reduced. Accordingly, in obtaining multiple images or a stream of images, the frame rate can be increased. For example, in some embodiments, the scan sequence 600 can provide an approximately 2× increase in frame rate compared to the full aperture sequence 300 shown in FIG. 5.

In some embodiments, fewer or more transmit-receive pairs can be skipped compared to the sequence 600 shown in FIGS. 8A and 8B. Accordingly, scan sequences can advantageously provide for controllable frame rates. In particular, scan sequences described in the present disclosure allow for increased frame rates while mitigating reduction in image quality. FIG. 9 illustrates a scan sequence 700 comprising fewer transmit-receive pairs compared to the sequence 600 shown in FIGS. 8A and 8B. In that regard, similar to the sequence 600 of FIGS. 8A and 8B, the sequence 700 comprises apertures 710, 720, 730 that each span fourteen transmit elements and fourteen receive elements. Each transmit element is associated with two transmit-receive pairs, the two transmit-receive pairs corresponding to two receive elements. Thus, the scan sequence 700 can be described as a “two-diagonal” scan sequence. The scan sequence 700 may, in some embodiments, provide an approximately 7× increase in frame rate compared to the full aperture sequence 300 shown in FIG. 5. It will be understood that it may be desirable for a scan sequence to comprise, for each transmit element, transmit-receive pairs that are associated with at least two receive elements. For example, scan sequences that comprise only one transmit-receive pair associated with each transmit element or receive element may not provide sufficient information to generate a satisfactory or coherent image. Accordingly, the sequence 700 is an example of a highly efficient scanning sequence. The sequence 700 may use a theoretical minimum number of transmit receive events for each aperture (2N−1), and only the pairs where abs(i−j)=0, 1 are preserved, maximizing both redundancy and SNR. All other sequences described in this disclosure may be less efficient, but may have other desirable attributes in some circumstances.

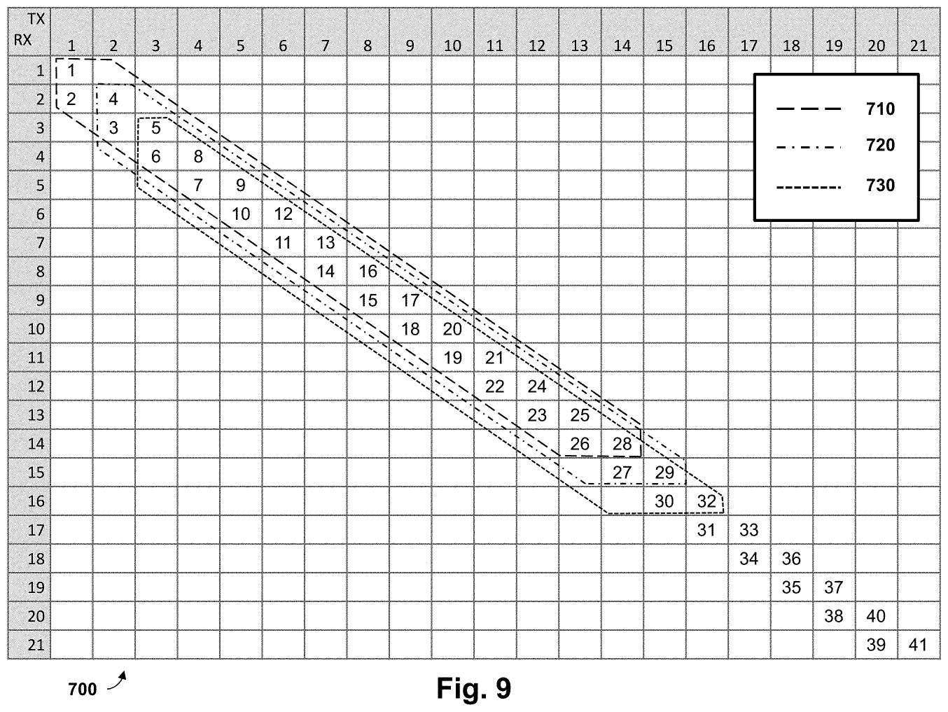

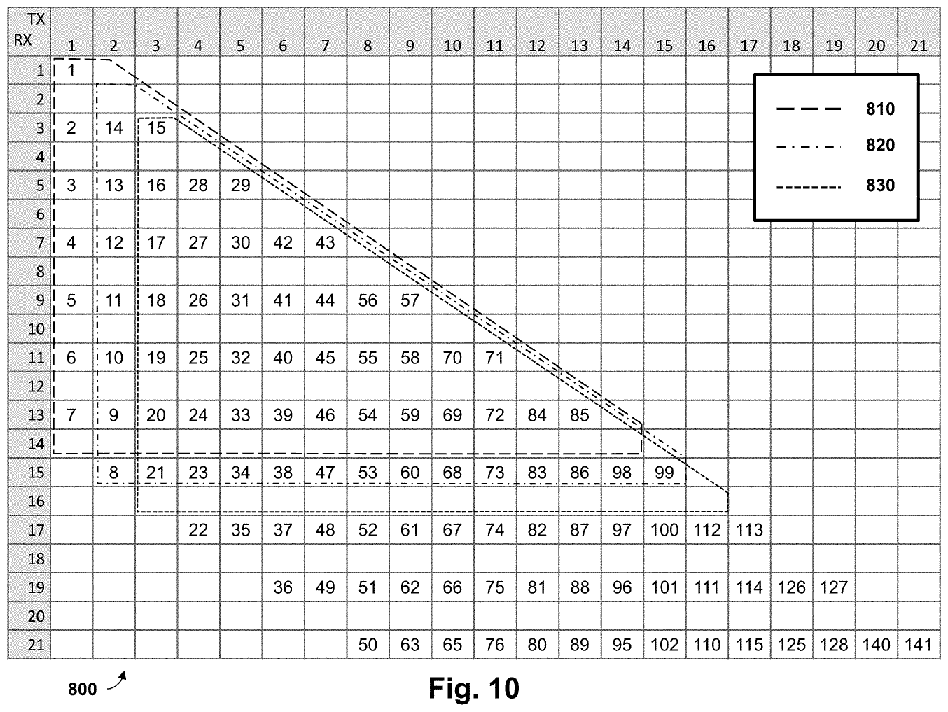

While the scan sequences 600 and 700 comprise contiguous groups of transmit-receive pairs in which all transmit elements in an aperture are activated to transmit, and all receive elements in the aperture are activated to receive, other scan sequences are also contemplated by the present disclosure that provide for controllable frame rates. FIG. 10 illustrates a scan sequence 800 comprising a plurality of triangularly-arranged apertures 810, 820, 830 in which a plurality of non-contiguous transmit-receive pairs are skipped. In particular, the scan sequence 800 comprises activating only odd receive elements and activating all transmit elements in an aperture. In other words, each aperture 810, 820, 830 comprises transmit-receive pairs associated with each of fourteen transmit elements, and only the even-indexed receive elements, amounting to seven receive elements. Accordingly, the scan sequence 800 can provide for an approximately 2× increase in frame rate compared to the sequence 300 shown in FIG. 5.

It will be understood that, while the even receive elements are skipped in the sequence 800, other embodiments are also contemplated in which similar principles are applied. For example, in some embodiments, the odd receive elements are skipped. In other embodiments, all receive elements are activated, but a portion of the transmit elements are not activated. For example, in some embodiments, transmit-receive pairs corresponding to even-indexed transmit elements are skipped while all receive elements are activated. In other embodiments, transmit-receive pairs corresponding to odd-indexed transmit elements are skipped while all receive elements are activated. In still other embodiments, transmit-receive pairs associated with a non-contiguous portion of transmit elements are skipped, and transmit-receive pairs associated with a non-contiguous portion of receive elements are also skipped.

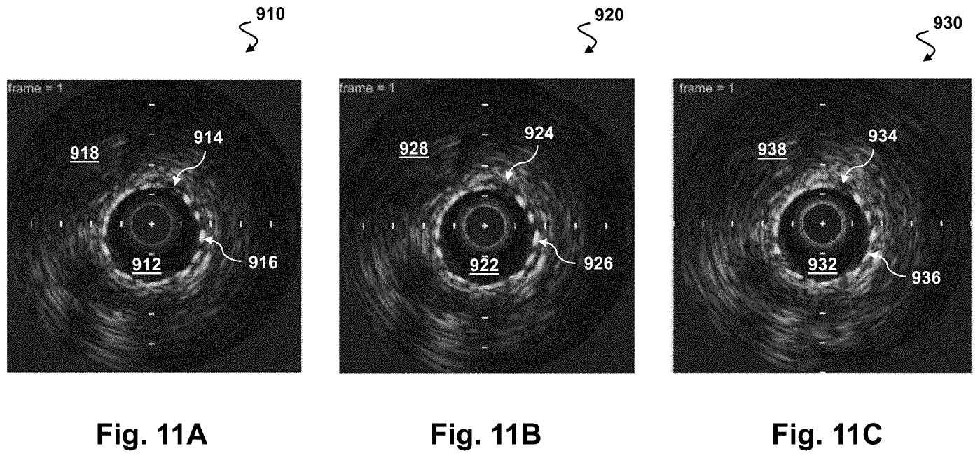

FIGS. 11A, 11B and 11C are IVUS images of a blood vessel obtained using, respectively, a full aperture, fourteen-diagonal scan sequence (as in FIG. 5), a seven-diagonal scan sequence (as in FIGS. 8A and 8B), and a two-diagonal scan sequence (as in FIG. 9). Accordingly, FIG. 11A is representative of an IVUS image obtained using the most transmit-receive pairs of the three images. The image 910 shows various features, including a lumen region 912, a vessel wall region 914, stent struts 916, and a tissue speckle region 918. The contrast and clarity of the image 910 allows the features corresponding to each region to be discerned.

FIG. 11B shows an image 920 obtained using a seven-diagonal scan sequence, as in FIGS. 8A and 8B. Accordingly, the image 920 can be obtained at a higher frame rate compared to the image 910 of FIG. 11A. Although there may be some differences in, for example, the tissue speckle region 918 when compared to the image 910 of FIG. 11A, the features of the blood vessel, including the lumen 912, vessel wall 914, stent struts 916, and tissue speckle region 918, are similarly identifiable in the image 920 of FIG. 11B. FIG. 11C shows an image 930 obtained using a two-diagonal sequence as in FIG. 9, and therefore represents a further increase in frame rate when compared to the image 920 of FIG. 11B. As in FIGS. 11A and 11B, the features of the blood vessel, including the lumen 932, vessel wall 934, stent struts 936, and tissue speckle region 938, are identifiable in the image 930 of FIG. 11C. Accordingly, embodiments of the present disclosure provide scan sequences in which controllable and increased frame rates can be achieved with little or no degradation of image quality.

It will be understood that one or more of the steps of the method 500, such as controlling the array to transmit and receive ultrasound energy, arranging the transmit-receive pairs into apertures, generating the image, and outputting the image to the display, can be performed by one or more components of an ultrasound imaging system, such as the processor, a multiplexer, a beamformer, a signal processing unit, an image processing unit, or any other suitable component of the system. For example, activating a scan sequence may be carried out by the processor 150 described with respect to FIG. 1B. The processor 150 may be in communication with a multiplexer configured to select or activate one or more elements of an ultrasound transducer array. In some embodiments, generating the ultrasound images may include beamforming signals from the ultrasound imaging device and processing the beamformed signals by an image processor. The processing components of the system can be integrated within the ultrasound imaging device, contained within an external console, or may be a separate component.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system for ultrasound imaging, comprising:
an array of acoustic elements configured to transmit ultrasound energy into an anatomy and receive echoes associated with the transmitted ultrasound energy; and
a processor circuit in communication with the array and configured to:
activate a pulse sequence comprising a plurality of transmit-receive pairs associated with a plurality of transmit elements and a plurality of receive elements of the array;
arrange the plurality of transmit-receive pairs into an aperture, the aperture spanning the plurality of transmit elements and the plurality of receive elements, wherein at least one of:
transmit-receive pairs corresponding to each transmit element correspond to only a portion of the plurality of receive elements; or
transmit-receive pairs corresponding to each receive element correspond to only a portion of the plurality of transmit elements;
generate an image using signals corresponding to the plurality of transmit-receive pairs arranged into the aperture; and
output the image to a display in communication with the processor.

2. The system of claim 1, wherein the processor is configured to arrange the plurality of transmit-receive pairs into the aperture by beamforming signals associated with the plurality of transmit-receive pairs.

3. The system of claim 2, wherein the processor is configured to arrange the plurality of transmit-receive pairs into the aperture by applying a weight to signals associated with each transmit-receive pair, wherein the applied weight is based on a distance between a transmit element and a receive element associated with the transmit-receive pair.

4. The system of claim 1, wherein the processor is configured to activate the pulse sequence by activating the plurality of transmit-receive pairs and skipping one or more nonactivated transmit-receive pairs, wherein the one or more nonactivated transmit-receive pairs correspond to a lower signal-to-noise ratio than the plurality of transmit-receive pairs activated by the processor.

5. The system of claim 4, wherein the one or more nonactivated transmit-receive pairs span a range of spatial frequencies, and wherein the plurality of transmit-receive pairs activated by the processor span at least the range of spatial frequencies.

6. The system of claim 4, wherein the processor is configured to:
activate the pulse sequence by activating each of the plurality of transmit-receive pairs more than once; and
arrange the plurality of transmit-receive pairs into an aperture comprises averaging duplicate transmit-receive pairs.

7. The system of claim 1, wherein the processor is configured to activate the pulse sequence by skipping one or more transmit-receive pairs and their spatial complements.

8. The system of claim 1, further comprising an intravascular ultrasound (IVUS) imaging catheter, and wherein the array of acoustic elements is positioned around a perimeter of the IVUS imaging catheter.

9. The system of claim 1, wherein the aperture comprises a contiguous group of transmit-receive pairs.

10. The system of claim 1, wherein the aperture comprises a non-contiguous group of transmit-receive pairs.

11. A method for ultrasound imaging, comprising:
transmitting, by an array of acoustic elements, ultrasound energy into an anatomy;
receiving, by the array, echoes associated with the transmitted ultrasound energy;
activating, by a processor circuit in communication with the array, a pulse sequence comprising a plurality of transmit-receive pairs associated with a plurality of transmit elements and a plurality of receive elements of the array;
arranging, by the processor, the plurality of transmit-receive pairs into an aperture, the aperture spanning the plurality of transmit elements and the plurality of receive elements, wherein at least one of:
transmit-receive pairs corresponding to each transmit element correspond to only a portion of the plurality of receive elements; or
transmit-receive pairs corresponding to each receive element correspond to only a portion of the plurality of transmit elements;
generating, by the processor, an image using signals corresponding to the plurality of transmit-receive pairs arranged into the aperture; and
outputting the image to a display in communication with the processor.

12. The method of claim 11, wherein arranging the plurality of transmit-receive pairs into the aperture comprises beamforming signals associated with the plurality of transmit-receive pairs.

13. The method of claim 12, wherein arranging the plurality of transmit-receive pairs into the aperture comprises applying a weight to signals associated with each transmit-receive pair, wherein the applied weight is based on a distance between a transmit element and a receive element associated with the transmit-receive pair.

14. The method of claim 11, wherein activating the pulse sequence comprises:
activating the plurality of transmit-receive pairs; and
skipping one or more nonactivated transmit-receive pairs, wherein the one or more nonactivated transmit-receive pairs correspond to a lower signal-to-noise ratio than the plurality of transmit-receive pairs activated by the processor.

15. The method of claim 14, wherein the one or more nonactivated transmit-receive pairs span a range of spatial frequencies, and wherein the plurality of transmit-receive pairs activated by the processor span at least the range of spatial frequencies.

16. The method of claim 14, wherein:
activating the pulse sequence comprises activating each of the plurality of transmit-receive pairs more than once; and
arranging the plurality of transmit-receive pairs into the aperture comprises averaging duplicate transmit-receive pairs.

17. The method of claim 11, wherein activating the pulse sequence comprises skipping one or more transmit-receive pairs and their spatial complements.

18. The method of claim 11, wherein arranging the plurality of transmit-receive pairs into the aperture comprises arranging a contiguous group of transmit-receive pairs into the aperture.

19. The method of claim 11, wherein arranging the plurality of transmit-receive pairs into the aperture comprises arranging a non-contiguous group of transmit-receive pairs into the aperture.

20. A system for ultrasound imaging, comprising:
an array of acoustic elements configured to transmit ultrasound energy into an anatomy and receive echoes associated with the transmitted ultrasound energy; and
a processor circuit in communication with the array and configured to:
activate a pulse sequence comprising a plurality of transmit-receive pairs associated with a plurality of transmit elements and a plurality of receive elements of the array;
arrange the plurality of transmit-receive pairs into an aperture, wherein the aperture spans N transmit elements and N receive elements, and wherein the aperture comprises fewer than half of N(N+1) transmit-receive pairs;
generate an image using signals corresponding to the plurality of transmit-receive pairs arranged into the aperture; and
output the image to a display in communication with the processor.
